(19) United States
(12) Patent Application Publication
(10) Pub. No.: US 2025/0221772 A1
(43) Pub. Date: Jul. 10, 2025

(54) 3-DIMENSIONAL TRACKING AND NAVIGATION SIMULATOR FOR NEURO-ENDOSCOPY

(71) Applicants: ALL INDIA INSTITUTE OF MEDICAL SCIENCES (AIIMS), New Delhi (IN); INDIAN INSTITUTE OF TECHNOLOGY DELHI, New Delhi (IN); DBT- DEPARTMENT OF BIOTECHNOLOGY, GOVT. OF INDIA, New Delhi (IN)

(72) Inventors: Britty BABY, New Delhi (IN); Ashish SURI, New Delhi (IN); Chetan ARORA, New Delhi (IN); Subhashis BANERJEE, New Delhi (IN); Prem Kumar KALRA, New Delhi (IN); Subodh KUMAR, New Delhi (IN); Ramandeep SINGH, New Delhi (IN)

(21) Appl. No.: 18/845,310

(22) PCT Filed: Mar. 9, 2023
(86) PCT No.: PCT/IB2023/052244

§ 371 Date: Sep. 9, 2024

(30) Foreign Application Priority Data
Mar. 9, 2022 (IN) 202211012683

(51) Int. Cl. A61B 34/10; A61B 34/20; A61B 90/00; G09B 23/28; G09B 23/34

(52) U.S. Cl. CPC A61B 34/10; A61B 34/20; A61B 90/37; A61B 90/39; G09B 23/285; G09B 23/34; A61B 2034/101; A61B 2034/2057; A61B 2034/2065; A61B 2090/3937; A61B 2090/3983

(57) ABSTRACT

A 3-dimensional tracking and navigation simulator system for image guided neuro-endoscopy, said system comprising: a fiducial based physical model of an anatomic region associated with neuro-endoscopy, wherein the physical model is mapped with CT/MRI slices of the anatomy; a virtual model of the anatomic region associated with neuro-endoscopy; at least one surgical instrument operable to perform neuro-endoscopy, said at least one surgical instrument is associated with a 3-D tracking component; a camera unit for tracking a location of the at least one surgical instrument with the 3-D tracking component, relative to a reference unit of the physical model; one or more displays for displaying the location of the at least one surgical instrument in the CT/MRI slices and endoscopic output, a computer work-station operably coupled to the physical model, and the camera unit, said work-station comprising: a localization and tracking module having configured to track the tasks performed by a user with the at least one surgical instrument, and estimate the position and rotation of the at least one surgical instrument in 6 degrees of freedom; a virtual space mapping module configured to map a location to a corresponding anatomic position in the virtual model to give a navigation of the surgical instrument in the virtual model; an evaluation module configured to evaluate the 3D tracking of tasks performed based on a localization metric and a 3D tracking metric, and provide feedback to the user based on the performance using machine learning and AI based algorithms.

### TECHNICAL FIELD

The present invention relates to a surgical training simulator and, more particularly, to a 3-Dimensional, 3D, tracking and navigation simulator for skills training of neuro-endoscopic surgeries along with image guidance and skills evaluation.

### BACKGROUND OF THE INVENTION

Minimally invasive surgical interventions are highly dependent on technology. Neurosurgery is a surgical specialty where it is more critical and indisputable. Neuro-navigation systems are assisting systems for image-guided neurosurgery for localization and delineation of tumors and aids in their accurate surgical resection. The basic idea is to locate the tip of a pointer in any given digital image space (CT/MRI), by mapping the device space to the image space. There is a transformation matrix that maps the anatomical space/device space with the digital image space (CT/MRI) determined by registration and calibration of the navigation device. The existing neuro-navigation systems are based on stereotactic frames, optical tracking methods or electro-magnetic tracking methods. The commercially available neuro-navigation systems are very expensive and are not available for training under laboratory circumstances. Neuro-navigation is frequently used in minimally invasive neuro-endoscopic procedures such as endoscopic endo-nasal skull base surgeries.

Neuro-endoscopy is a type of neurosurgery where the surgeons use an endoscope to navigate through the brain. The long-shafted instruments and the different perspective vision of the surgery make it challenging for the neurosurgeons to get accustomed. The learning curve is steep and challenging under the apprenticeship model of learning. Also, with the work hour constraints and demanding medical care, learning a surgical procedure and corresponding skills on patients is not advisable. Simulation-based learning has been the solution for this, and simulators vary from natural simulations to highly equipped virtual reality simulations.

The physical simulators are more suitable to develop psychomotor skills. However, no physical simulator provides training of image-guided navigation of anatomical structures. Simulators that would make the neurosurgeons equipped with the real surgical scenario should include orientation for navigating the vital anatomical structures. Simulators provide a safe-environment to learn and re-learn. With objective evaluation involved in the simulators, it provides the trainees feedback on their performance and specific ways to improve the skills.

Existing prior art does not provide simulations for neuro-endoscopy that provide training for image guidance. Skills training systems that can be used as simulators and for anatomical orientation is lacking in the current scenario of simulators.

In this regard, reference is made to U.S. Pat. No. 10,417,936B2 disclosing methods and systems for clinical procedure training via mixed reality simulators. There is a physical model and a virtual model of an anatomical region. It also includes a tracking component that tracks any one instrument, an anatomical feedback component, which reproduces the changes in the physical model based on the interaction between instrument and virtual model. It can detect electrical signals, pressure and flow. The feedback to the physical model depends on tracked position of the clinical device. The physical model and virtual model is co-registered for the better performance and created out of same anatomical source like CT, MRI. It provides procedure simulation for nerve-blocks. The physical simulator is fitted with robotic actuators. The material selection of physical model is based on realistic appearance, texture and haptic feedback. The instruments used are purely mechanical or electronic, and as the user manipulates the instrument, spatial tracking component utilizes 3D tracking system and map those positions to virtual model. The instrument has one or more sensors to monitor 5 DOF (Degree of Freedom) and the data is transmitted wired or wireless manner. The location/tracking can be determined by electromagnetic tracking system or ultrasound probes.

Reference is made to US20180042514A1 disclosing a system and apparatus that is taught to determine the identification of the instrument involved in the surgery to help in navigation. This includes the information being fed into a RFID reader by the user at a selected time. This information can be used to assist the navigation of an instrument in relative to a patient. The navigation system is used to track instruments like catheters, needles, guide wires, instruments, can allow surgeon to view on a display a relative position of an instrument to a coordinate system. The coordinate system can be made relative to the image, such as in image guided procedure and can be registered to a patient only. Image data can be captured and forwarded to the navigation computer. A surgical navigation system includes navigating a procedure related to an anatomy, tracking device associated with the instrument, tracking system to track the location of the device, identity of the instrument based on selected information, and RFID tag-based identification of the instrument and tracking. It can also be used for multiple instruments.

Reference is made to US20180125604A1 teaching a navigation method and system relative to anatomy, using an instrument, identifying the position of the instrument, and navigating the instrument related to the anatomy. It includes imaging the anatomy, displaying the working portion of the instrument in relation to the image data by electromagnetic tracker. The projection and receiving portion using electromagnetic field and can be used for multiple instruments. The tracking system includes an electromagnetic localizer and is operable to obtain data from an identification member. There is a communication system that interconnects the tracking system and the navigation processor. The navigation processor determines the identification of the surgical instrument based on the data transferred to the navigation processor from the identification member.

Reference is made to JP2020106844A disclosing medical training equipment and methods for training in minimally invasive surgery. It includes a medical instrument, medical device structure positioned in relative to the physical simulation area, display device to visualize the virtual surgical site which is a simulated surgical view. The medical device is manipulated with a manipulator arm and the surgical instrument is positioned in the virtual environment with respect to a reference arm. It provides feedback to the user comparing the position of the manipulator arm in comparison to the reference arm. The simulation area is manipulated based on the position of the medical device. In the simulated surgery, the virtual environment is updated based on the physical interaction and the position is mapped by manipulator arm.

Yet another reference is made to RU2726476C1 teaching methods for accelerated training of otorhinolaryngologists for basic skills of endoscopic endonasal surgery by Sinus Model Otorhino-Neuro Trainer simulator (S.I.M.O.N.T). It is a simulation model for providing skills training for sinus surgery. It helps in practicing skills on a biological material made of silicone and helps in repeated practice in the laboratory. It provides hands-on coordination training using instrument and endoscope.

Reference is further made to WO2012106706A2 disclosing systems and methods facilitating training in clinical procedures via mixed reality simulations. Such a system can comprise a physical model and a virtual model of an anatomic region associated with the procedure, wherein the virtual model associates tissue types with locations in the physical model. The system can include a tracking component that tracks locations of at least one clinical instrument relative to the models, and an anatomic feedback component that can produce perceptible changes in the physical model based on the interaction between the instrument and virtual model. A clinical device interface can detect outputs of clinical devices like electrical signals, pressure or flow, wherein feedback to the physical model depends on the tracked position of a clinical device and output from the same or different clinical device. Another component can generate feedback effects to the clinical device. Aspects can simulate anesthesiology procedures like local nerve blockade.

WO2012106706A2 is a mixed reality simulation model that involves physical and virtual model for anesthesiology that simulate local nerve blockade of arm. Whereas the present invention is a navigation-based simulation and training system that maps the physical model relevant to neuro-endoscopy to the CT/MRI of the corresponding anatomical region and not only the virtual model of the region. The visualization and training motive for both the simulators are distinctly different. The training simulator of the present invention involves training of the neuro-endoscopic surgeons for navigating the brain and regions of the skull as they usually do inside the operating room. The visualization provided is also identical to the navigation systems. Whereas in the present invention, the simulation is intended for procedure-based skills training to provide realistic physical models to train with the help of image guidance.

The tracking component in WO2012106706A2 is concerned with a commercially available electromagnetic tracking system to locate the instrument and it is susceptible to interference with the nearby powered systems and instruments. In the present invention, the tracking methodology, is based on a single-camera and AruCo-marker based tracking system that maps the location of the tip of the instrument to the 3D CT space of the respective physical model. The camera-based tracking system provides the pose of the instrument (Rotation and Translation) with respect to a reference AruCo-tracker. The tracking system is robust to any powered inferences.

The anatomic feedback component in WO2012106706A2 produces changes in the physical model based on the interaction of the instrument in the virtual model. Whereas, in the present invention, the feedback mechanism works differently. The physical feedback is direct as the trainee uses clinical instruments for the training and the visual and haptic feedback is directly received with the help of the endoscope and the physical model respectively. The tracking and visualization with the 3D CT planes are an additional information and cue to the surgeon to locate themselves and learn where they are anatomically and manipulate with precise movements. The present invention provides a user interface similar to the operation theatre.

WO2012106706A2 includes the tracking information to be used to provide simple feedback to the trainee like time, velocity. In the present invention, the 3D positions of the tool and the endoscopic camera inputs are captured and stored for feedback on the skills of the performance. These sensor data are synchronized so that the input can be analyzed using deep-learning based skills evaluation technique to provide specific skills feedback to the surgeon in terms of Likert-scale like skills evaluation metric. Our invention includes image-guidance/navigation and Artificial Intelligence/Deep-learning based skills evaluation of the surgery. The present invention develops algorithms to rate the surgeons based on global rating scale for the endoscopic video input and 3D tracking information for the task performed on the simulator.

Reference is further made to US20100167253A1 disclosing a surgical simulator which includes at least one tracking system configured to track movement within a body form, including movement of an instrument located partially within the body form, and a computer receiving data from the tracking system and outputting data to a display. A display displays a virtual background including a virtual image of at least a portion of the instrument, virtual movement of the instrument, and a virtual image of at least one organ. The simulator also includes a physical model corresponding to the at least one organ and located within the body form, the physical model providing haptic feedback when contacted by the instrument.

US20100167253A1 includes non-anatomical physical models for providing haptic feedback. Our invention includes anatomically relevant physical models for training of neurosurgeons. The one-tracking system mentioned in US20100167253A1 includes the instrument to be located partially within the body form and the trackers are present inside the body form. Whereas, in the present invention, the tracking system is present outside the trainer and it can also be extended to surgical scenario without any dependency of a casing or body form.

The instrument can freely move and no need to be fixed with the body form. US20100167253A1 displays the virtual background including the portion of the instrument, it's movement and image of the organ. In the present invention, the visualization includes CT/MRI slices and the location/tip of the instrument is mapped to the CT space. The physical model included in US20100167253A1 is non-anatomical and there is no relevant anatomical mapping to the virtual model. In the present invention, the same CT/MRI used for visualization is segmented, and reproduced as physical model to replicate the exact anatomy for training. The present invention further includes image-guidance/navigation and Artificial Intelligence/Deep-learning based skills evaluation of the surgery.

Reference is also made to US20100159434A1 which provides mixed simulator systems that combine the advantages of both physical objects/simulations and virtual representations. In-context integration of virtual representations with physical simulations or objects can facilitate education and training. Two modes of mixed simulation are provided. In the first mode, a virtual representation is combined with a physical simulation or object by using a tracked display capable of displaying an appropriate dynamic virtual representation as a user moves around the physical simulation or object. In the second mode, a virtual representation is combined with a physical simulation or object by projecting the virtual representation directly onto the physical object or simulation. In further embodiments, user action and interaction can be tracked and incorporated within the mixed simulator system to provide a mixed reality after-action review. The subject mixed simulators can be used in many applications including, but not limited.

US20100159434A1 is a mixed simulator that combines the physical and virtual representations. The present invention is a navigation-based simulation and training system that maps the physical model to the CT/MRI of the corresponding anatomical region and not the virtual representation. US20100159434A1 includes the tracking using optical sensors (Infrared cameras) and there are two modes of visualization. One where the virtual representation dynamically moves as the user moves and it is displayed. In the second mode, the virtual representation is projected to the physical simulation. In the present invention, the visualization mode is the CT space of the physical model and the tip of the instrument inside the physical model is mapped to the relevant anatomical structure. The tracking of US20100159434A1 is focused mainly towards after-action review. In the simulator of the present invention, the tracking is focused on the image guidance to the trainee and as a 3D tracking tool to capture the movements for skills evaluation. US20100159434A1 does not include any specific feedback for the trainee for improvement of skills and is a generalized module. The present invention is a dedicated simulator for skills evaluation in neurosurgery and provide feedback to the trainee with the help of our custom-designed deep learning module, and includes image-guidance/navigation and Artificial Intelligence/Deep-learning based skills evaluation of the surgery.

Yet another reference is made to WO2014116278A1, the embodiments disclosing a surgical training system (100) comprising an anatomical training model (102), three or more telemetry sensors (104) attached to the training model, a training tool (106), a display (112), and a controller (108). The anatomical training model physically simulates human anatomical features. The training tool comprises at least one transmitter (122) configured to emit a signal. The controller includes one or more processors configured to determine a location of the training tool relative to the training model using the sensors in the signal, and produce a virtual image of the training tool and anatomical features simulated by the training model on the display based on the location of the training tool. In embodiments of a surgical training method, a training tool (106) is positioned (160) near an anatomical training model (102), which physically simulates human anatomical features. A position of the training tool relative to the training model is determined (162) using a controller (108). An orientation of the training tool relative to the training model is determined (164) using the controller. A virtual tool (130), which corresponds to the training tool, is located (166) within a virtual model (128), which corresponds to the training model, based on the position and orientation of the training tool using the controller. The virtual tool and the virtual model are displayed (168) on a display (112) using the controller.

WO2014116278A1 includes surgical training system simulating the human anatomical structures for a physician to practice an electrode implantation without the need for a cadaver. In the present invention, simulation training is focused towards the surgical training with the exact anatomical features of the CT/MRI of the patient. The simulation is intended for procedure-based skills training to provide realistic physical models to train with the help of image guidance. WO2014116278A1 involves use of telemetry sensors attached to the training model. In the present invention, the tracker of light weight is attached to any one of the surgical tools and there is no requirement camera to be present inside the training model. The camera can be placed outside to track the tool tip with no requirement of the tool tip to be visible explicitly. In WO2014116278A1, the orientation of the training tool relative to the training model is determined using a controller for display purpose. In the present invention, the training tool is 3D tracked with the help of AruCo-marker and the tool tip is projected to the CT/MRI space of the anatomical structure. The inputs of the endoscopic camera and the camera tracking the tool are also captured for skills evaluation. In WO2014116278A1, there is no specific feedback provided to the trainee on their performance. In the present invention, feedback is provided to the trainee depending upon their performance. It includes image-guidance/navigation and Artificial Intelligence/Deep-learning based skills evaluation of the surgery.

In addition to the above cited prior art, there are commercially available Neuro-navigation system that work with the help of stereotactic frames, optical tracking methods or electro-magnetic tracking methods, to locate a specific probe being inserted to the brain. These systems are very costly and not available for training in the laboratory. The commercially available neuro-endoscopic simulators are synthetic simulators for hands-on procedural skills training. They do not provide any objective metric for skills evaluation. The integrated system that provides image guided surgery training, neuro-endoscopic procedural skills training along with objective evaluation is missing in the neurosurgical skills training scenario.

Therefore, in view of the exiting prior art, there is a dire need for a neuro-endoscopic surgery training module with 3D tracking and low-cost navigation system for training neurosurgeons for image-guided surgery along with endoscopic simulation training.

### SUMMARY OF THE INVENTION

The following presents a simplified summary of the invention in order to provide a basic understanding of some aspects of the invention. This summary is not an extensive overview of the present invention. It is not intended to identify the key/critical elements of the invention or to delineate the scope of the invention. Its sole purpose is to present some concept of the invention in a simplified form as a prelude to a more detailed description of the invention presented later.

An object of the present disclosure is to provide a neuro-endoscopic surgery training module with 3D tracking and low-cost navigation system for training neurosurgeons for image-guided surgery along with endoscopic simulation training and artificial intelligence-based skills evaluation.

An object of the present disclosure is to provide a neuro-endoscopic surgery training module with 3D tracking and low-cost navigation system integrating computer vision techniques to track ArUco based markers using a machine vision camera unit and localize the tip of surgical instrument in the CT/MRI space of a human anatomical model.

An object of the present disclosure is to provide a method for image guidance, anatomical and endoscopic orientation of neurosurgeons and a machine learning and deep-learning based method for evaluating the endoscopic skills based on the performance. It includes a neuro-endoscopic skills training simulator that provides 3D tracking of the trainee along with objective evaluation metric of the performed task.

An object of the present disclosure is to provide a neuro-endoscopic multi-purpose simulator for image-guided neurosurgery and neuro-endoscopic simulation with 3D tracking based skills evaluation, and develop algorithms to rate the surgeons based on global rating scale for the endoscopic video input and 3D tracking information for the task performed on the simulator.

A first aspect of the present invention, a 3-dimensional tracking and navigation simulator system for image guided neuro-endoscopy, said system comprising: a fiducial based physical model of an anatomic region associated with neuro-endoscopy, wherein the physical model is mapped with CT/MRI slices of the anatomy; a virtual model of the anatomic region associated with neuro-endoscopy; at least one surgical instrument operable to perform neuro-endoscopy, said at least one surgical instrument is associated with a 3-D tracking component; a camera unit for tracking a location of the at least one surgical instrument with the 3-D tracking component, relative to a reference unit of the physical model; one or more displays for displaying the location of the at least one surgical instrument in the CT/MRI slices and endoscopic output; a computer work-station operably coupled to the physical model, and the camera unit, said work-station comprising: a localization and tracking module having configured to track the tasks performed by a user with the at least one surgical instrument, and estimate the position and rotation of the at least one surgical instrument in 6 degrees of freedom (DOF); a virtual space mapping module configured to map a location to a corresponding anatomic position in the virtual model to give a navigation of the surgical instrument in the virtual model; an evaluation module configured to evaluate the 3D tracking of tasks performed based on a localization metric and a 3D tracking metric, and provide feedback to the user based on the performance using machine learning and AI based algorithms.

Other aspects, advantages, and salient features of the invention will become apparent to those skilled in the art from the following detailed description, which, taken in conjunction with the annexed drawings, discloses exemplary embodiments of the invention.

### BRIEF DESCRIPTION OF ACCOMPANYING DRAWINGS

The above and other aspects, features and advantages of the embodiments of the present disclosure will be more apparent in the following description taken in conjunction with the accompanying drawings, in which:

FIG. 1 illustrates a schematic diagram depicting the 3D tracking and Navigation simulator for Neuro-endoscopy, according to an embodiment of the present invention.

FIG. 2 illustrates a schematic diagram of the 3D physical simulation anatomical models for training, according to an embodiment of the present invention.

FIG. 3 illustrates a schematic diagram of 3D tracker design of the instrument with tool tracker with ArUco marker attached to the biopsy forceps, according to an embodiment of the present invention.

FIG. 4 illustrates a detailed block diagram 3D tracking and navigation simulator for neuro-endoscopy, according to an embodiment of the present invention.

FIG. 5 illustrates anatomical points defined in the skull for the experiment, according to an embodiment of the present invention.

FIG. 6 illustrates a setup with a neurosurgeon practicing on the simulator system, according to an embodiment of the present invention.

Persons skilled in the art will appreciate that elements in the figures are illustrated for simplicity and clarity and may have not been drawn to scale. For example, the dimensions of some of the elements in the figure may be exaggerated relative to other elements to help to improve understanding of various exemplary embodiments of the present disclosure. Throughout the drawings, it should be noted that like reference numbers are used to depict the same or similar elements, features, and structures.

### DETAILED DESCRIPTION OF THE INVENTION

The following description with reference to the accompanying drawings is provided to assist in a comprehensive understanding of exemplary implementations of the invention. It includes various specific details to assist in that understanding but these are to be regarded as merely exemplary.

Features that are described and/or illustrated with respect to one implementation may be used in the same way or in a similar way in one or more other implementations and/or in combination with or instead of the features of the other implementations.

Neuro-endoscopy is a surgical specialty that demands bimanual, visual, cognitive and psychomotor skills. The endoscopic surgeries are performed with the help of a 2-Dimensional display and the surgeon creates the mental picture of a 3D anatomy using the depth cues. In neuro-endoscopic surgeries, the tumour resection is aided with the help of image guidance to accurately localize the tumour and remove it. But there are no specific training simulators based on navigation/image guidance, or neuro-endoscopic simulation along with anatomical CT orientation. There are some commercially available neuro-endoscopic synthetic simulators but there is no image guidance-based training and they do not provide 3D tracking of the surgeon's activity.

Embodiments of the present invention disclose a multi-purpose simulator for neuro-endoscopic surgical skills simulation, skills evaluation using 3D tracking and image guidance.

Various embodiments of the present invention describe a 3D Tracking and Navigation Simulator for Neuro-Endoscopy (3DTN Simulator). These embodiments include one or more of the components to provide the 3DTN Simulator. It includes a physical anatomical model, virtual model of the anatomy, registration software to map the physical model with CT/MRI slices of the anatomy or a virtual model, 3D tracking of any instrument used for surgery fitted with our tracker, calibration of the coordinate system with respect to physical and virtual space, navigation of the instrument in the virtual anatomy, localization of the anatomical structure, simulation environment for neuro-endoscopic surgery, 3D tracking of the tasks performed, evaluation of the tasks based on localization metric and 3D tracking metric, feedback to the trainee based on the performance using machine learning and AI based algorithms.

In the following description, for purpose of explanation, specific details are set forth in order to provide an understanding of the present disclosure. The term “module” used in the application refers to a computer-related unit, either hardware, or a combination of hardware and software.

The term “feedback” used in the application includes physical feedback directly provided as the trainee using clinical instruments for the training and/or the visual and haptic feedback received with the help of the endoscope and the physical model. It also includes feedback provided as in the form of a synopsis of their activity including the objective metric regarding their performance and specific feedback on their skill level.

This involves training of the neuro-endoscopic surgeons for navigating the brain and regions of the skull similar to the operating room. The visualization provided is identical to the navigation/image-guidance systems. This is intended for procedure-based skills training on physical models with realistic visual and haptic feedback, with the help of image guidance and feedback module for individual development of specific skills.

According to an embodiment, 3-Dimensional tracking and navigation simulator for neuro-endoscopy includes a camera unit, surgical instrument-based tracker, reference unit, fiducial based physical simulation model, it's CT/MRI scan and software for process of registration of virtual to physical model, 3D tracking of any neurosurgical instrument fitted with our tracker, 3 plane CT/MRI visualization, evaluation software for surgical skills analysis. The software for registration of CT/MRI images with the physical simulator has been developed. The model can be replaced by any simulation model of importance like ETV simulators, spine surgery simulators or general surgery simulators.

In one of the embodiments, the present invention is related to 3DTN simulator with fiducial based anatomically relevant physical simulation model of neuro-endoscopic surgery with its corresponding CT/MRI scan. This anatomical model along with fiducials have been CT/MRI scanned with XY resolution of 0.60 mm and Z-axis resolution of 1 mm placed according to the simulation requirement.

Another embodiment is a platform to place the anatomical models for different training scenario. This platform includes housing for the camera unit, reference unit and also the anatomical models. It also includes fixtures to fasten the models and accessories to cover the model for training.

Yet another embodiment, include a localization and tracking module with a tri-planar tool tracker made of planes at different angles with each other that can be fitted to any neurosurgical instrument. The tracker is stuck with ArUco markers. An ArUco marker is a synthetic square marker composed of a wide black border and an inner binary matrix that determines its identifier (id). The tri-planar structure is designed to estimate at least 2 planes from any viewpoint. The ArUco markers with different identifiers were stuck to determine the different planes. The tri-planar tracker is modeled and the 3D points of the corners were known with respect to an origin.

Yet another embodiment includes, a reference tracker using 2 planes placed at different angles apart from one another.

Another embodiment includes a camera unit for tracking ArUco markers. The camera is placed on a platform in such a way that in a given frame, both the tool tracker and reference unit are visible.

Yet another embodiment is a physical localization module that collects the data from the vision camera, and localize the tool tip with respect to the reference unit. This module provides the spatial location and pose of the instrument. The tracking module then track the tool-tip frame by frame and is robust to any powered interferences. The localization and tracking module can monitor the instrument or tool at every given time. This includes 6 degrees of freedom (DOF). There is no wired sensor attached to the tool and hereby no interference to the surgeon's activity.

Yet another embodiment is the virtual space mapping module that registers the tool tip instrument of the physical space with respect to the virtual space and localizes the tip on the CT/MRI slices. The position of the tip of the tool estimated with respect to the reference coordinate system and is projected onto the CT/MRI of the simulation model for visualization and maps to the respective anatomical location. The simulation model is assumed to be fitted with fiducial marker and a corresponding CT/MRI coordinate system associated with it. The CT/MRI data were resampled to 0.60 mm (XY resolution) in all three directions and a new set of CT/MRI images were created using the VTK library. The CT/MRI points were made to visualize on all the three planes; axial, coronal and sagittal. Another embodiment is localization of the anatomical structure in the virtual space and 3D model.

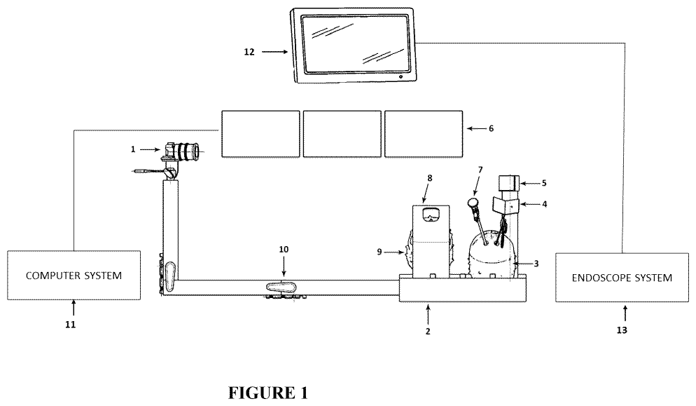

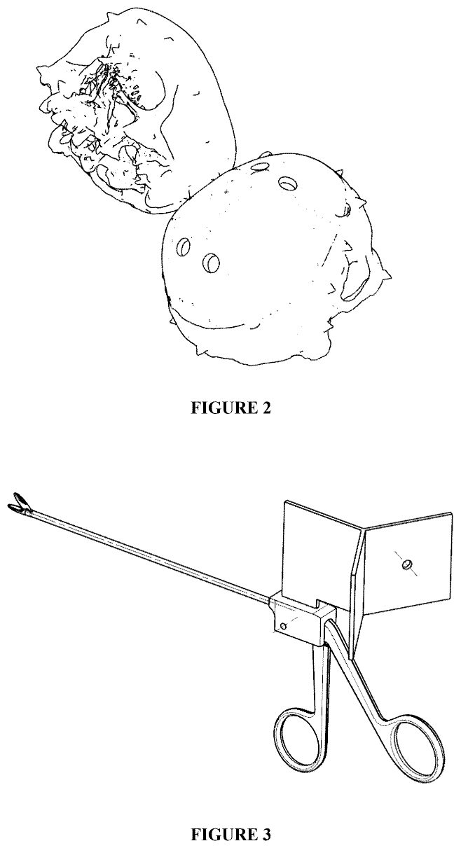

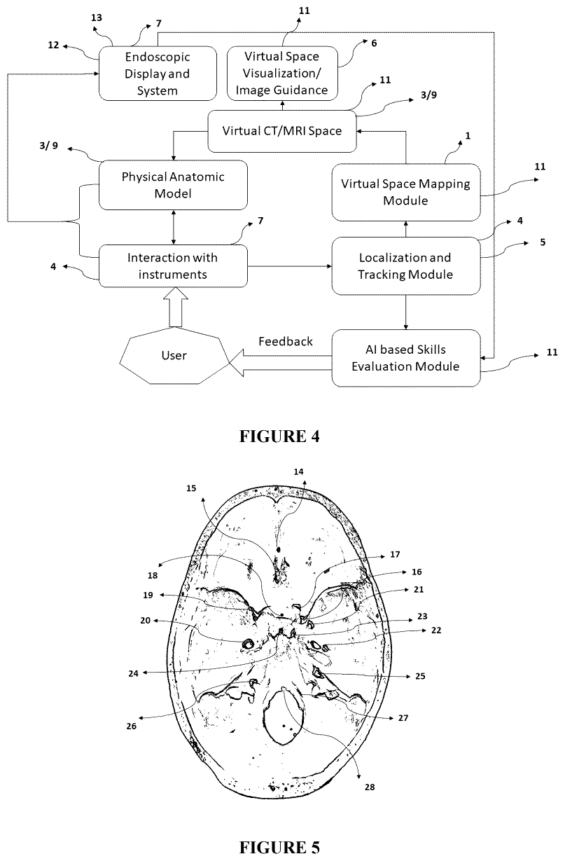

An embodiment is a simulator system comprising one or more displays for displaying the location of at least one surgical instrument in the CT/MRI slices and an endoscopic output (6, 12), as shown in FIG. 1. Further, the system comprises a computer work-station (11) operably coupled to the physical model, and the camera unit (1), and the work-station comprises: a localization and tracking module having configured to track the tasks performed by a user with at least one surgical instrument, and an endoscope (7) and estimate the position and rotation of at least one surgical instrument in 6 degrees of freedom (DOF); a virtual space mapping module configured to map a location to a corresponding anatomic position in the virtual model to give a navigation of the surgical instrument in the virtual model; and an evaluation module configured to evaluate the 3D tracking of tasks performed based on a localization metric and a 3D tracking metric, and provide feedback to the user based on the performance using machine learning and AI based algorithms.

Another embodiment is the synthetic simulation environment for neuro-endoscopic surgery with any 3D printed physical/synthetic model like skull/skin/dura/brain/tumor structures with replaceable modules after training.

Another embodiment is the method of training that includes the data collected according to the surgical simulation experiment like identification of anatomical structures, sellar drilling, and pituitary tumor resection.

Another embodiment is a method to validate the usefulness of the developed system by establishing face, content, construct, and concurrent validity measures. The activity was to use the 3D tracking tool and start from a reference point placed outside the simulator and reach the anatomical structure inside the simulator using endoscope on one hand and tool on the other hand.

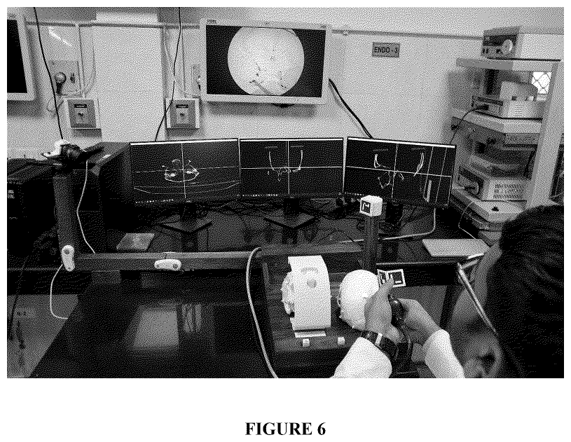

Yet another embodiment is to identify anatomical structures of relevance to train the anatomy and endoscopic handling simultaneously (FIG. 5).

Another embodiment is the localization metric and 3D tracking metric for the evaluation of the neuro-endoscopic skills. Another embodiment is the endoscopic camera input that is synchronized with the 3D tracking to provide another sensor information for the skills evaluation. 3D information of the tool and the endoscopic camera inputs are captured and stored for feedback on the skills of the performance. These sensor data are synchronized and provided as input to Artificial Intelligence (AI) based skills evaluation module.

Another embodiment is the Artificial Intelligence (AI) based skills evaluation module that includes automatic evaluation of the skills using machine-learning and deep-learning based algorithms to segment and track the instruments and estimate the level of expertise of the trainee in the form of an objective Likert-scale like metric with correlation to the subjective evaluation of an expert. The tracking data and the endoscopic video output can be utilized for expert evaluation. It can also be used by the trainee to evaluate their own earlier performance and can be accessed using a web interface or on request. The web module can include interaction by the trainee and also by the experts for evaluation. Another embodiment is the interface to provide the personalized specific feedback and skills scoring to the trainee based on the performance and level of skills.

Another embodiment is the usage of the AI based skills evaluation module directly for the surgery video analysis as it also includes specific paths and embedding that can be modified for the real-surgical scenario for evaluation of skill levels. It can include clearance of field and interaction with the background as part of the evaluation.

According to an embodiment, the single simulator act as an image guidance, 3D tracking and localization of the instrument in CT/MRI slices and virtual model and for skills evaluation of the performance. It provides training for neuroendoscopy along with anatomical orientation, CT visualization, anatomy of related structures and hands-on skills of procedure-based neuro-endoscopic skills. The 3D tracking leads to accurate estimation of pose, localization of the instrument while manipulating and helps in evaluation of the surgeon by 6 degree of freedom data and differentiate the performance of the expert and novice. This happens with a light-weight tracker attached to one or more instruments wirelessly. This tracking device also helps the accurate localization in the CT/MRI and thereby identify the exact location of reach by different users. This invention includes image-guidance/navigation and Artificial Intelligence/Deep-learning based skills evaluation of the skills of the trainee. It includes segmenting and tracking the instruments and automatic skills evaluation using objective Likert-scale and specific feedback on the individual skill level. This evaluation module includes specific paths and embedding that can be modified for the real-surgical scenario for evaluation of surgical skills.

The system developed is made independent of the position of the camera, by providing an external reference. The camera also tracks the external reference and estimates the rotation and translation of it with respect to the camera center. We then map the coordinate geometry to obtain the position of the tracker attached at the rear end of the surgical tool with respect to the external reference.

Some of the non-limiting advantages of the 3D Tracking and Navigation Simulator for Neuro-Endoscopy are:
1. Multi-purpose simulator for simulation of neuro-endoscopic surgery along with image guidance, 3D tracking of the instrument, and machine learning/AI based skills evaluation.
2. Utilized for development of hands-on skills and visual orientation for neuro-endoscopic surgeries along with the image-guided system and provides training for neuro-navigation/image guidance along with skills evaluation with the help of 3D XYZ coordinate-based tracking of the surgeon's activity. Allows development of procedure-based skills along with CT and 3D visualization of the relevant anatomical structures.
3. Objective evaluation of the surgeon's activity based on the virtual localization metric and 3D tracking metric based on exact localization of the anatomical structure along with evaluation parameters like hand-eye coordination, instrument-tissue manipulation, dexterity, flow of procedure and effectualness.
4. 3D model can be obtained from the CT/MRI of human anatomical structures and the CT visualization involves 3 planes (axial, coronal, and sagittal).
5. Provision of 3D tracking and evaluation based on XYZ coordinates. It can also be incorporated with other sensors. It can also be extended to real-surgical scenario.

Although a 3D Tracking and Navigation Simulator for Neuro-Endoscopy for skills training of neuro-endoscopic surgeries along with image guidance and skills evaluation and a method thereof has been described in language specific to structural features and/or methods, it is to be understood that the implementations disclosed in the above section are not necessarily limited to the specific features or methods or devices described. Rather, the specific features are disclosed as examples of implementations of the 3D Tracking and Navigation Simulator for Neuro-Endoscopy for skills training of neuro-endoscopic surgeries along with image guidance and skills evaluation and the method thereof.

### REFERENCE NUMERALS

1) Camera Unit
2) Platform for fixation
3) Fiducial-fitted simulation model 1
4) Tool tracker
5) Reference unit
6) Screens for visualization
7) Endoscope
8) Flap/Accessories for simulation model
9) Fiducial-fitted simulation model 2
10) Fixtures for the platform
11) Computer work-station/System
12) Endoscopic Display
13) Endoscope System
14) Foramen Cecum,
15) Left Cribriform Plate,
16) Right Anterior Clinoid Process Tip,
17) Right Optic Canal,
18) Tuberculum Sellae Midpoint,
19) Left Superior Orbital Fissure lower border,
20) Left Foramen Ovale,
21) Right Foramen Rotundum,
22) Right Foramen Spinosum,
23) Right Posterior Clinoid Process,
24) Dorsum Sellae Midpoint,
25) Right Internal Auditory Canal,
26) Left Jugular Foramen,
27) Right Hypoglossal Foramen,
28) Midpoint of Anterior Border of Foramen Magnum.

1. A 3-dimensional tracking and navigation simulator system for image guided neuro-endoscopy, said system comprising:
a fiducial based physical model of an anatomic region associated with neuro-endoscopy, wherein the physical model is mapped with CT/MRI slices of the anatomy;
a virtual model of the anatomic region associated with neuro-endoscopy;
at least one surgical instrument operable to perform neuro-endoscopy, said at least one surgical instrument is associated with a 3-D tracking component;
a camera unit for tracking a location of the at least one surgical instrument with the 3-D tracking component, relative to a reference unit of the physical model;
one or more displays for displaying the location of the at least one surgical instrument in the CT/MRI slices and endoscopic output a computer work-station operably coupled to the physical model, and the camera unit, said work-station comprising:
a localization and tracking module having configured to track the tasks performed by a user with at least one surgical instrument, and an endoscope and estimate the position and rotation of the at least one surgical instrument in 6 degrees of freedom;
a virtual space mapping module configured to map a location to a corresponding anatomic position in the virtual model to give a navigation of the surgical instrument in the virtual model;
an evaluation module configured to evaluate the 3D tracking of tasks performed based on a localization metric and a 3D tracking metric, and provide feedback to the user based on the performance using machine learning and AI based algorithms.

2. The simulator system as claimed in claim 1, wherein the tracking component is a tri-planar tool tracker made of planes at different angles with each other operably fitted to the at least one surgical instrument, and the tracker is stuck with ArUco markers.

3. The simulator system as claimed in claim 1, includes the synthetic simulation environment for neuro-endoscopic surgery with any 3D printed physical/synthetic model like skull/skin/dura/brain/tumor structures with replaceable modules after training.

4. The simulator system as claimed in claim 1, wherein the camera unit is configured to track ArUco markers, and the camera is mounted on a platform in such a way that in a given frame, both the tool tracker and reference unit are visible.

5. The simulator system as claimed in claim 1, wherein the localization and tracking module is configured to: collect data from the camera, and localize the surgical instrument tip with respect to the reference unit, to provide spatial location and pose of the surgical instrument.

6. The simulator system as claimed in claim 1, wherein the virtual space mapping module is configured to register the tool tip of the surgical instrument of the physical space with respect to the virtual space, and localize the tip on the CT/MRI slices, and the position of the tip of the tool estimated with respect to the reference coordinate system is projected onto the CT/MRI of the virtual model for visualization and maps to the respective anatomical location, wherein the CT/MRI are visualized on all three planes-axial, coronal and sagittal.

7. The simulator system as claimed in claim 1, includes the data collected according to the surgical simulation experiment like identification of anatomical structures, sellar drilling, and pituitary tumor resection with respect to the activity performed by the user.

8. The simulator system as claimed in claim 1, wherein the system is configured to give a visual and haptic feedback received with the help of the endoscope and the physical model.

9. The simulator system as claimed in claim 1, wherein an input from an endoscopic camera is synchronized with the 3D tracking to provide sensor information for the evaluation module; and 3D information of the tool and the endoscopic camera inputs are captured and stored in the workstation, for feedback and score on the skills of the tasks performed by the user.

10. The simulator system as claimed in claim 1, wherein evaluation module is an artificial intelligence based skills evaluation module configured to automatically evaluate the skills using machine-learning and deep-learning based algorithms to segment and track the instruments and estimate the level of expertise of the user in the form of an objective Likert-scale like metric with correlation to the subjective evaluation of an expert.

11. A method for 3-dimensional tracking and navigation for image guided neuro-endoscopy in a simulator system as claimed in claim 1.
